(19) United States
(12) Patent Application Publication
(10) Pub. No.: US 2025/0327833 A1
(43) Pub. Date: Oct. 23, 2025

(54) METHOD FOR DETERMING POSITION OF BODY AND METHOD FOR SECURING DATA ON PATIENT'S PHYSICAL ACTIVITY SIGNS USING SAME

(71) Applicant: LUXNINE CORPORATION, Seoul (KR)

(72) Inventors: In Ho KIM, Seoul (KR); Jong Hun KIM, Seoul (KR)

(73) Assignee: LUXNINE CORPORATION, Seoul (KR)

(21) Appl. No.: 18/872,153

(22) PCT Filed: Jun. 7, 2023
(86) PCT No.: PCT/KR2023/007727

§ 371 Date: Dec. 5, 2024

(30) Foreign Application Priority Data
Jun. 7, 2022 (KR) 10-2022-0069036

(51) Int. Cl. G01P 15/18; G06F 3/01; G06F 17/16

(52) U.S. Cl. CPC G01P 15/18; G06F 3/011; G06F 17/16

(57) ABSTRACT

In a method for determining body position a sensor attached within a pentagon-cuboid-superimposed space is used to calculate acceleration or angular velocity values for each of three axis directions in an absolute coordinate system. An opposite direction of gravity is defined as a Z-axis direction, a front view direction in patient's standing position is defined as an X-axis direction, and a Y-axis direction is defined as a direction that generates the Z-axis through vector product with the X-axis direction. Sensor inclination values for each of the three axis directions of the absolute coordinate system are obtained using the acceleration or angular velocity values. Corrected sensor inclination values are derived by correcting the sensor inclination values using sensor's own inclination value that changes according to the sensor's attachment position when the patient is standing, and the patient's position is determined from the corrected sensor inclination values, wherein the pentagon-cuboid-superimposed space is defined as an interior of a pentagon connecting patient's left and right pectoralis major apices, both shoulder deltoid apices and chin.

### CROSS-REFERENCE TO RELATED APPLICATIONS

The present application is a National Stage Application of PCT International Application No.: PCT/KR2023/007727 filed on Jun. 7, 2023, which claims priority to Korean Patent Application 10-2022-0069036, filed with the Korean Patent Office on Jun. 7, 2022, the disclosures of which are incorporated herein by reference in their entirety.

### BACKGROUND

Example embodiments of the present invention relate to a method for determining a position of body and a method for securing data on patient's physical activity signs using the same. More specifically, example embodiments of the present invention relate to a method for easily determining patient's body position when measuring body data and a method for securing data on patient's physical activity signs using the method for determining the body position.

Recently, with the dramatic development of information and communication technology and significant changes in social awareness of healthcare, efforts are being made to establish a medical system that transitions from treatment-centered medicine to prevention-centered medicine, and from disease management-centered healthcare to health management-centered healthcare through the combination of information communication technology and healthcare technology.

In particular, electronic devices incorporating information communication technology, such as wearable watches, provide body data including body temperature, pulse, blood oxygen pressure, and electrocardiogram. As these body data accumulate, vital signs that can confirm changes in the user's body can be detected.

However, when these electronic devices measure body data, the reliability of the body data may be compromised as the data varies depending on measurement conditions. For example, the measurement conditions for an electrocardiogram test are “the patient should lie in a supine position (or semi-sitting position), remain still during the examination, and should remove any metal from their body [Source: Cardiology Department ECG Measurement Manual].” Therefore, for medically meaningful ECG measurements when there is no metal on the patient's body, first, the patent should maintain his body to supine, semi-sitting, or semi-Fowler's position, and second, it should be confirmed that the patent is sufficiently stable. Thus, in a medical environment, body data needs to be measured with the patient in a specified position.

In medical settings, patient positions are broadly classified into twelve types: supine position, prone position, lateral position, dorsal recumbent position, lithotomy position, knee-chest position, Sims' position, Fowler's position, semi-Fowler's position, Trendelenburg position, reverse Trendelenburg position, and jackknife position.

Specifically, hospitalized patients' positions should be maintained comfortably close to basic anatomical positions, with joints slightly flexed to prevent increased muscle tension and fatigue, and when changing positions, the normal range of motion (ROM) of joints should be observed. For patients at risk of pressure sores, positions should be changed every two hours.

All stages of position selection, change, maintenance, and recording are performed only by medical personnel, and there is no method to automatically determine these states.

However, recently there has been an increasing need for accumulating healthcare data based on various IoT devices. In particular, vital signs such as body temperature, pulse, blood oxygen pressure, and electrocardiogram are the most valuable human body data. For these body data to be useful in non-face-to-face situations or telemedicine settings, information about the subject's body position at the time of measurement is essential.

Therefore, although technology for automatically determining nursing patients' positions is essential for diagnosis in telemedicine environments where direct assistance from medical personnel is not available, no solution has been presented for this until now.

### SUMMARY

Example embodiments of the present invention provide a method for automatically determining body positiony.

Example embodiments of the present invention provide a method for securing data on patient's physical activity signs with automatically determining body position.

According to example embodiments of the present invention, a method for determining body position includes using a sensor fixed within a pentagon-cuboid-superimposed space to calculate acceleration or angular velocity values for each of three axis directions in an absolute coordinate system, where an opposite direction of gravity is defined as a Z-axis direction, a front view direction in patient's standing position is defined as an X-axis direction, and a Y-axis direction is defined as a direction that generates the Z-axis through vector product with the X-axis direction, obtaining sensor inclination values for each of the three axis directions of the absolute coordinate system using the acceleration or angular velocity values, deriving corrected sensor inclination values by correcting the sensor inclination values using sensor's own inclination value that changes according to the sensor's attachment position when the patient is standing, and determining the patient's position from the corrected sensor inclination values, wherein the pentagon-cuboid-superimposed space is defined as an interior of a pentagon connecting patient's left and right pectoralis major apices, both shoulder deltoid apices and chin.

In an example embodiment of the present invention, the sensor may be attached to patient's anterior mediastinum.

In an example embodiment of the present invention, the sensor may include an accelerometer or gyroscope sensor.

In an example embodiment of the present invention, when the sensor is an accelerometer, an X-axis direction acceleration value is defined as αx, a Y-axis direction acceleration value is defined as αy, a Z-axis direction acceleration value is defined as αz, and acceleration measurement values for each of the X-axis through Z-axis directions are defined as a vector R\vec{ }, the Y-axis direction acceleration value αy satisfies following Mathematical Formulas 1 and 2;

$$\begin{matrix}9.8 = {\sqrt{\vert \vec{{α}_{X}} + \vec{{α}_{Y}} + \vec{{α}_{Z}} \vert}}^{2} & Mathematical Formula 1 \\ ∠ ( \vec{{α}_{X}} + \vec{{α}_{Y}} + \vec{{α}_{Z}} ) = - ∠ \vec{z} , & Mathematical Formula 2\end{matrix}$$

and the sensor inclination values are defined by Mathematical Formula 3;

$${θ}_{X} r = arccos ( Rx / R ) , \\ {θ}_{Y} r = arccos ( R τ / R ) , \\ {θ}_{Z} r = arccos ( Rz / R ) \qquad Mathematical Formula 3$$

where θ_{XT}, θ_{YT} and θ_{ZT} are the sensor inclination values in the X-axis, Y-axis, and Z-axis directions, respectively, R_{X}, R_{Y}, and R_{Z} are the magnitudes of R\vec{ } in each direction, and R is the magnitude of R\vec{ }.

In an example embodiment of the present invention, correcting the sensor inclination values using sensor's own inclination value that changes according to the sensor's attachment position may include aligning a z-axis direction of sensor's local coordinate system, which varies according to the sensor's attachment position, with the Z-axis direction.

In an example embodiment of the present invention, correcting the sensor inclination values using sensor's own inclination value that changes according to the sensor's attachment position may include performing a matrix transformation using following Mathematical Formula 4:

$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$

where θ_{XT}, θ_{YT}, and θ_{ZT} are the sensor inclination values for the X-axis, Y-axis, and Z-axis directions, respectively, θ_{XT}′, θ_{YT}′, and θ_{ZT}′ are the corrected sensor inclinations values for the X-axis, Y-axis, and Z-axis directions, respectively, and β is a rotation angle required to align the z-axis direction of sensor's local coordinate system with the Z-axis direction of the absolute coordinate system when the patient is standing, rotating counterclockwise.

According to example embodiments of the present invention, a method for securing data on patient's physical activity signs, includes confirming a presence of a preliminary vital sign signal from the patient, when the preliminary vital signal is present, determining the patient's position; and when the patient's position is qualified, measuring patient's main vital sign signals and transmitting the main vital sign signal, wherein determining the patient's position includes using a sensor fixed within a pentagon-cuboid-superimposed space to calculate acceleration or angular velocity values for each of three axis directions in an absolute coordinate system, where an opposite direction of gravity is defined as a Z-axis direction, a front view direction in patient's standing position is defined as an X-axis direction, and a Y-axis direction is defined as a direction that generates the Z-axis through vector product with the X-axis direction, obtaining sensor inclination values for each of the three axis directions of the absolute coordinate system using the acceleration or angular velocity values, deriving corrected sensor inclination values by correcting the sensor inclination values using sensor's own inclination value that changes according to the sensor's attachment position when the patient is standing, and determining the patient's position from the corrected sensor inclination values, wherein the pentagon-cuboid-superimposed space is defined as an interior of a pentagon connecting patient's left and right pectoralis major apices, both shoulder deltoid apices and chin.

According to the example embodiments of the present invention described above, a patient's body position can be defined using the acceleration values of the sensor that change according to the sensor's attachment position on the patient. As a result, a reliability of the vital signs data according to the patient's position can be improved.

### BRIEF DESCRIPTION OF THE DRAWINGS

The above and other features and advantages of the present invention will become more apparent by describing in detailed example embodiments thereof with reference to the accompanying drawings, in which:

FIG. 1 is a photograph defining the X-axis, Y-axis, and Z-axis directions and the pentagon-cuboid-superimposed space based on the patient's standing position;

FIG. 2 is a flowchart illustrating a method for determining body position according to example embodiment of the present invention;

FIG. 3 is a schematic diagram explaining an example of determining a semi-sitting position;

FIG. 4 is a coordinate system for explaining how to calculate sensor inclination values using acceleration measurements;

FIG. 5 is a coordinate system for explaining the directions according to sensor's own local coordinate system;

FIG. 6 is a schematic diagram for explaining the step of correcting the sensor inclination values using the sensor's own inclination value; and

FIG. 7 is a flowchart illustrating a method for securing data on patient's physical activity signs according to example embodiments of the present invention.

### DESCRIPTION OF THE EMBODIMENTS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the sizes and relative sizes of layers and regions may be exaggerated for clarity.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

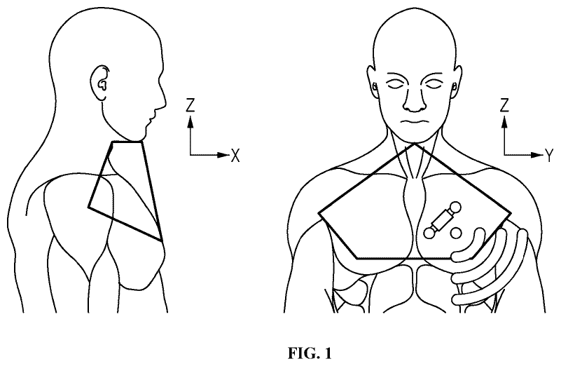

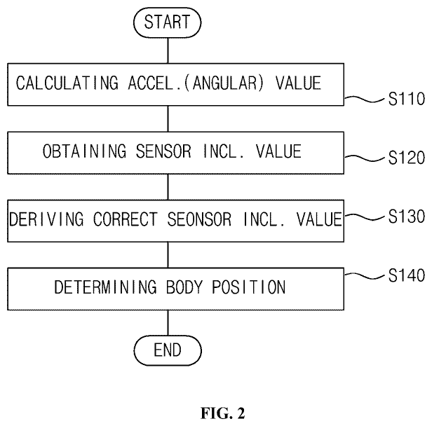

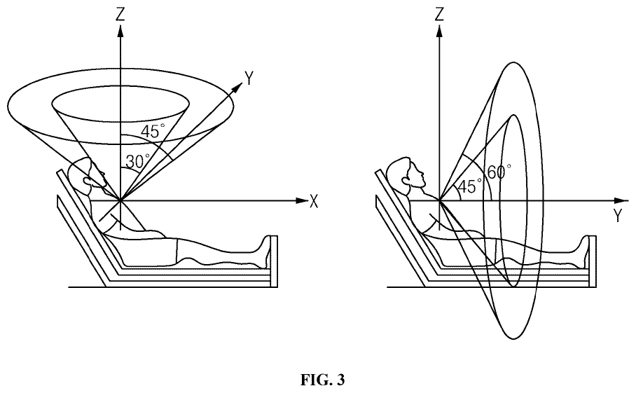

FIG. 1 is a photograph defining the X-axis, Y-axis, and Z-axis directions and the pentagon-cuboid-superimposed space based on the patient's standing position. FIG. 2 is a flowchart illustrating a method for determining body position according to example embodiment of the present invention. FIG. 3 is a schematic diagram explaining an example of determining a semi-sitting position.

Referring to FIG. 1 to FIG. 3, disclosed is a method for determining body position in accordance with some example embodiments of the present invention. First, a sensor fixed within patient's pentagon-cuboid-superimposed space is used. An opposite direction of gravity is defined as a Z-axis direction, a front view direction in patient's standing position is defined as an X-axis direction, and a Y-axis direction is defined as a direction that generates the Z-axis through vector product with the X-axis direction. Thus, three axis directions are defined according to an absolute coordinate system.

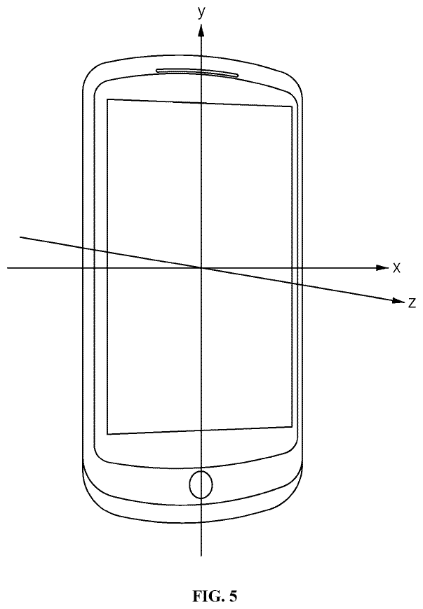

Meanwhile, regarding sensor's own local coordinate system, three axis directions including x-axis direction, y-axis direction, and z-axis direction are defined in the local coordinate system. The local coordinate system will be described later with reference to FIG. 5.

In this case, acceleration or angular velocity values for each of the three axis directions of the absolute coordinate system are calculated (S110).

Here, the pentagon-cuboid-superimposed space is defined as the interior of a pentagon connecting patient's left and right pectoralis major apices, both shoulder deltoid apices, and chin. In particular, the sensor may be attached to patient's anterior mediastinum.

The pentagon-cuboid-superimposed space corresponds to an area of patient's body, having the least degree of freedom. That is, even when the patient moves, the sensor attached within the pentagon-cuboid-superimposed space may secure reliable data compared to when attached to other parts, for example, the limbs.

Additionally, even when patient' is in a lying position, the sensor does not press against patient's body, thus not causing discomfort to the patient.

When the sensor includes an accelerometer, it is easy to distinguish commonly used medical positions such as supine position, semi-sitting position (Fowler's position), and semi-Fowler's position. Furthermore, when the sensor includes a gyroscope, it can determine whether the lateral position is right-sided or left-sided, which can help reduce the burden of sleep apnea.

Meanwhile, the Z-axis direction and X-axis direction of the absolute coordinate system can be specified as absolute directions. Furthermore, the Y-axis direction can be defined as the direction that generates the Z-axis direction through vector product with the X-axis direction. In FIG. 1, the Y-axis direction may correspond to a direction connecting the patient's shoulder apices to each other.

The sensor may include, for example, an accelerometer or gyroscope sensor. In the case of the accelerometer, acceleration value may be measured, and in the case of the gyroscope sensor, angular velocity value may be measured.

Meanwhile, patient positions are broadly classified into twelve types: supine position, prone position, lateral position, dorsal recumbent position, lithotomy position, knee-chest position, Sims' position, Fowler's position, semi-Fowler's position, Trendelenburg position, reverse Trendelenburg position, and jackknife position.

Regarding the patient's position, the examination areas and application situations can be summarized as shown in Table 1 below.

TABLE 1

| Position | Examination Area | Application Situation |
|---|---|---|
| Supine Position | Axilla, heart, abdomen, pulse | Male catheterization, spinal fracture, before |
| | measurement | consciousness recovery after surgery, ECG |
| | | measurement |
| Semi-sitting | — | Dyspnea, consciousness recovery after |
| Position | | surgery |
| Knee-chest Position | Rectum, rectoscopy | Fetal position correction, menstrual pain |
| | | relief, prevention of uterine retroversion, fetal |
| | | survival after umbilical cord prolapse |
| Lithotomy Position | Female reproductive organs, | Delivery |
| | cervical cancer examination, | |
| | cystoscopy | |
| Dorsal Recumbent | Abdominal examination | Female catheterization, abdominal hernia |
| Position | (pressure removal) | |
| Sims' Position | Rectum, vagina | Endoscopic examination |
| (lateral) | | |
| Prone Position | Pelvic joint extension | Post-diaphragmatic hemorrhage drainage |
| | | surgery, back massage |

Next, sensor inclination values for each of the three axis directions of the absolute coordinate system are calculated using the acceleration or angular velocity values (S120).

Here, when the sensor is an accelerometer, the X-axis direction acceleration value is defined as ax, the Y-axis direction acceleration value as ay, and the Z-axis direction acceleration value as az. At this time, the Y-axis direction acceleration value ay satisfies Mathematical Formulas 1 and 2 below.

$$\begin{matrix}9.8 = {\sqrt{\vert \vec{{α}_{X}} + \vec{{α}_{Y}} + \vec{{α}_{Z}} \vert}}^{2} & Mathematical Formula 1 \\ ∠ ( \vec{{α}_{X}} + \vec{{α}_{Y}} + \vec{{α}_{Z}} ) = - ∠ \vec{z} , & Mathematical Formula 2\end{matrix}$$

That is, the sensor receives a gravitational acceleration value of 9.8 m/s² in the direction toward the earth's center point, i.e., the gravity direction. Thus, if the sensor is attached to the chest of a standing patient and the patient is in a standing position, a 9.8 m/s² acceleration acts in the negative direction of the Z-axis, which is opposite to the gravity direction.

Therefore, using an accelerometer in a state where the patient does not move, the vector sum of the sensor's gravitational acceleration values always has a magnitude of 1G (9.8 m/sec²) and direction of −Z.

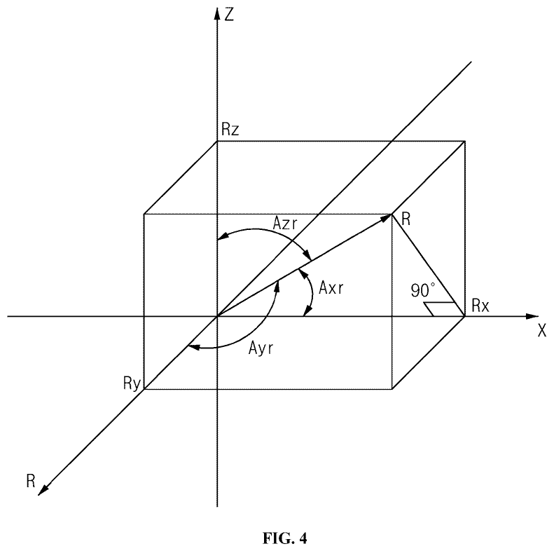

FIG. 4 is a coordinate system for explaining how to calculate sensor inclination values using acceleration measurements.

Referring to FIG. 4, the acceleration measurements for each of the X-axis through Z-axis directions are defined as vector R\vec{ }, and the sensor inclination values are defined by Mathematical Formula 3 below.

$${θ}_{X} r = arccos ( Rx / R ) , \\ {θ}_{Y} r = arccos ( R τ / R ) , \\ {θ}_{Z} r = arccos ( Rz / R ) \qquad Mathematical Formula 3$$

where θ_{XT}, θ_{YT} and θ_{ZT} are the sensor inclination values in the X-axis, Y-axis, and Z-axis directions, respectively, R_{X}, R_{Y}, and R_{Z} are the magnitudes of R\vec{ } in each direction, and R is the magnitude of R\vec{ }.

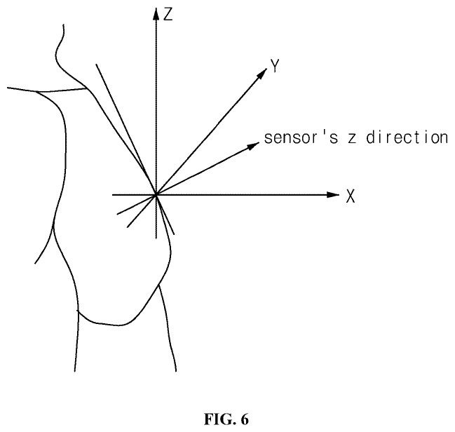

FIG. 5 is a coordinate system for explaining the directions according to the sensor's own local coordinate system. FIG. 6 is a schematic diagram explaining the step of correcting the sensor inclination values using the sensor's own inclination value.

Referring to FIG. 1, FIG. 5 and FIG. 6, corrected sensor inclination values are derived by correcting the sensor inclination values using the sensor's own inclination value that changes according to the sensor's attachment position (S130).

That is, measured gravity value can vary depending on the attachment surface, which is part of the patient's body. Therefore, it needs to be corrected using the sensor's own inclination value.

More specifically, to correct the sensor inclination values using the sensor's own inclination value when the patient is standing, the z-axis direction of the sensor's local coordinate system, which varies according to the sensor's attachment position, is aligned with the Z-axis direction of the absolute coordinate system.

For example, the sensor attached to the anterior mediastinum has its x-y plane as the circuit board surface where the chip is attached, corresponding to the attachment surface of the anterior mediastinum, and its z-axis direction extends perpendicular to the chip, normal to the attachment surface. Therefore, the local coordinate system of the sensor attached to the patient's pentagon-cuboid-superimposed space includes the x-axis, y-axis, and z-axis directions, which may cause be the local coordinate to differs from the absolute coordinate system based on the space with the Z-axis which corresponds to the vertical direction of the standing patient, where the absolute coordinate includes the X-axis, Y-axis, and Z-axis directions.

For example, a step of aligning sensor's z-axis direction with the Z-axis direction of the absolute coordinate system may involve rotating sensor's z-axis direction by 60 to 70 degrees. This is because the patient's chest is inclined relative to the gravity direction (negative Z-axis) by 20 to 30 degrees.

That is, corrected sensor inclination values (θXr′, θYr′, and θZr′) are derived through matrix transformation using Mathematical Formula 4 below;

$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$

Here, θ_{XT}, θ_{YT}, and θ_{ZT} are the sensor inclination values for the X-axis, Y-axis, and Z-axis directions, respectively, θ_{XT}′, θ_{YT}′, and θ_{ZT}′ are the corrected sensor inclinations values for the X-axis, Y-axis, and Z-axis directions, respectively, and β is a rotation angle required to align the z-axis direction of sensor's local coordinate system with the Z-axis direction of the absolute coordinate system when the patient is standing, rotating counterclockwise.

Subsequently, the patient's position is determined from the corrected sensor inclination values (S140).

The corrected sensor inclination values θXr′, θYr′, and θZr′ correspond to the corrected sensor inclinations for the X-axis, Y-axis, and Z-axis directions of the absolute coordinate system.

TABLE 2

| | X-axis Inclination | Y-axis Inclination | Z-axis Inclination |
|---|---|---|---|
| Position | Angle (θ_{X}r′, °) | Angle (θ_{Y}r′, °) | Angle (θ_{Z}r′, °) |
| Supine Position | 0 | 0 | 90(−90) |
| Semi-sitting Position | 45~60 | 0 | 30~45 |
| (Fowler's) | | | |
| Semi-Fowler's | 25~30 | 0 | 60~65 |
| Position | | | |
| Lateral Position | 90(−90) | 0 | 90(−90) |
| Prone Position | −180 | 90(−90) | 90(−90) |
| Trendelenburg | −10 | 0 | >100(−100) |
| Position | | | |

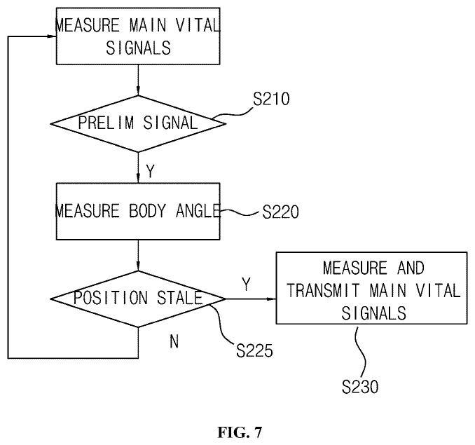

FIG. 7 is a flowchart illustrating a method for securing data on patient's physical activity signs according to example embodiments of the present invention.

Referring to FIG. 7, in the method for securing data on patient's physical activity signs according to example embodiments of the present invention, first, a presence of a preliminary vital signs signal from the patient is confirmed (S210). These preliminary vital signs may include biological vital signs unique to the human body, such as body temperature, respiration, pulse, blood oxygen saturation, and electrocardiogram, which are measured first and processed as a start command.

Subsequently, if a signal processed as the start command exists, the patient's position is determined by measuring body angles (S220). The qualification of the patient's position is then determined (S225). Afterward, the patient's main vital signs are measured, and the data related to these main vital signs is transmitted (S230).

To determine the patient's position, a sensor attached within the patient's pentagon-cuboid-superimposed space is used, where the gravity direction is defined as the Z-axis direction, and the front view direction in the patient's standing position is defined as the X-axis direction, to calculate acceleration or angular velocity for each of the three axis directions. Then, using this acceleration or angular velocity, the sensor's inclination values for each of the three axis directions are calculated. Corrected sensor inclination values are derived by correcting the sensor inclination values using the sensor's own inclination value based on its attachment position. Subsequently, the patient's position is determined from these corrected sensor inclination values. Here, the pentagon-cuboid-superimposed space is defined as the interior of a pentagon connecting the patient's left and right pectoralis major apices, both shoulder deltoid apices, and chin.

The device according to embodiments of the present invention may include a processor that performs each step constituting the body position determination method by processing data, a memory that stores program data, a permanent storage such as a disk drive, a communication port that communicates with external devices and user interface devices such as a touch panel, keys, buttons, etc. Methods implemented as software modules or algorithms can be stored as computer-readable codes or program instructions executable on the processor on a computer-readable recording medium. Computer-readable recording media may include magnetic storage media (e.g., ROM (read-only memory), RAM (random-access memory), floppy disks, hard disks, etc.), optical reading media (e.g., CD-ROMs, DVDs (Digital Versatile Discs)), and the computer-readable recording medium can be distributed among computer systems connected through a network, and computer-readable codes can be stored and executed in a distributed manner. The media is computer-readable, can be stored in memory, and can be executed by a processor.

The embodiments of the present invention can be represented by functional blocks and various processing steps. These functional blocks can be implemented by various numbers of hardware and/or software configurations that execute specific functions. For example, the embodiment may employ integrated circuit configurations such as a memory, a processor, a logic device, look-up tables, etc. which can execute various functions under the control of one or more microprocessors or other control devices. Similar to how components of the invention can be executed by software programming or software elements, embodiments can be implemented by various algorithms including combinations of data structures, processor, routines, other programming configurations implemented in programming or scripting languages such as C, C++, Java, assembler, etc. Functional aspects can be implemented as algorithms executed on one or more processors. Also, the embodiment may employ conventional technology for electronic configuration, signal processing, and/or data processing. Terms such as “mechanism”, “element”, “means”, “configuration” can be used broadly and are not limited to mechanical and physical configurations. These terms may include the meaning of a series of software routines in association with a processor, etc.

### INDUSTRIAL APPLICABILITY

The particular implementations described in this specification are examples and do not in any way limit the scope of the embodiment. For the sake of brevity in the specification, descriptions of conventional electronic configurations, control systems, software, and other functional aspects of the systems may be omitted. Furthermore, the connecting lines or connecting members between components shown in the drawings are intended to illustrate functional connections and/or physical or circuit connections by way of example and may be represented in actual devices by alternative or additional functional connections, physical connections, or circuit connections.

Finally, the specific implementations described in this specification are examples of implementing the technical idea of the present invention and various modifications are possible within the scope of the technical idea of the present invention. Therefore, although specific embodiments have been shown and described herein, various modifications and changes may be made without departing from the disclosed embodiments. The scope of the present invention is not limited by the foregoing description but is defined by the appended claims, and all differences within equivalent scope should be construed as being included in the present invention.

1. A method for determining body position comprising:
using a sensor fixed within a pentagon-cuboid-superimposed space to calculate acceleration or angular velocity values for each of three axis directions in an absolute coordinate system, where an opposite direction of gravity is defined as a Z-axis direction, a front view direction in patient's standing position is defined as an X-axis direction, and a Y-axis direction is defined as a direction that generates the Z-axis through vector product with the X-axis direction;
obtaining sensor inclination values for each of the three axis directions of the absolute coordinate system using the acceleration or angular velocity values;
deriving corrected sensor inclination values by correcting the sensor inclination values using sensor's own inclination value that changes according to the sensor's attachment position when the patient is standing; and
determining the patient's position from the corrected sensor inclination values,
wherein the pentagon-cuboid-superimposed space is defined as an interior of a pentagon connecting patient's left and right pectoralis major apices, both shoulder deltoid apices, and chin.

2. The method of claim 1, wherein the sensor is attached to patient's anterior mediastinum.

3. The method of claim 1, wherein the sensor includes an accelerometer or gyroscope sensor.

4. The method of claim 1, wherein when the sensor is an accelerometer:
an X-axis direction acceleration value is defined as αx,
a Y-axis direction acceleration value is defined as αY,
a Z-axis direction acceleration value is defined as αz,
acceleration measurement values for each of the X-axis through Z-axis directions are defined as a vector R\vec{ },
the Y-axis direction acceleration value ay satisfies following Mathematical Formulas 1 and 2:
$$\begin{matrix}9.8 = {\sqrt{\vert \vec{{α}_{X}} + \vec{{α}_{Y}} + \vec{{α}_{Z}} \vert}}^{2} & Mathematical Formula 1 \\ ∠ ( \vec{{α}_{X}} + \vec{{α}_{Y}} + \vec{{α}_{Z}} ) = - ∠ \vec{z} , & Mathematical Formula 2\end{matrix}$$
and the sensor inclination values are defined by Mathematical Formula 3:
$${θ}_{X} r = arccos ( Rx / R ) , \\ {θ}_{Y} r = arccos ( R τ / R ) , \\ {θ}_{Z} r = arccos ( Rz / R ) \qquad Mathematical Formula 3$$
where θXr, θYr and θZr are the sensor inclination values in the X-axis, Y-axis, and Z-axis directions, respectively, Rx, RY, and Rz are the magnitudes of R\vec{ } in each direction, and R is the magnitude of R\vec{ }.

5. The method of claim 1, wherein correcting the sensor inclination values using sensor's own inclination value that changes according to the sensor's attachment position comprises aligning a z-axis direction of sensor's local coordinate system, which varies according to the sensor's attachment position, with the Z-axis direction.

6. The method of claim 1, wherein correcting the sensor inclination values using sensor's own inclination value that changes according to the sensor's attachment position comprises performing a matrix transformation using following Mathematical Formula 4:
$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$
where:
θXr, θYr, and θZr are the sensor inclination values for the X-axis, Y-axis, and Z-axis directions, respectively,
θXr′, θYr′, and θZr′ are the corrected sensor inclinations values for the X-axis, Y-axis, and Z-axis directions, respectively, and
β is a rotation angle required to align the z-axis direction of sensor's local coordinate system with the Z-axis direction of the absolute coordinate system when the patient is standing, rotating counterclockwise.

7. A method for acquiring data on patient's physical activity signs, comprising:
confirming a presence of a preliminary vital sign signal from the patient;
when the preliminary vital signal is present, determining the patient's position; and
when the patient's position is qualified, measuring patient's main vital sign signals and transmitting the main vital sign signal,
wherein determining the patient's position includes:
using a sensor fixed within a pentagon-cuboid-superimposed space to calculate acceleration or angular velocity values for each of three axis directions in an absolute coordinate system, where an opposite direction of gravity is defined as a Z-axis direction, a front view direction in patient's standing position is defined as an X-axis direction, and a Y-axis direction is defined as a direction that generates the Z-axis through vector product with the X-axis direction;
obtaining sensor inclination values for each of the three axis directions of the absolute coordinate system using the acceleration or angular velocity values;
deriving corrected sensor inclination values by correcting the sensor inclination values using sensor's own inclination value that changes according to the sensor's attachment position when the patient is standing; and
determining the patient's position from the corrected sensor inclination values,
wherein the pentagon-cuboid-superimposed space is defined as an interior of a pentagon connecting patient's left and right pectoralis major apices, both shoulder deltoid apices and chin.
